(19) United States
(12) Patent Application Publication
(10) Pub. No.: US 2022/0193027 A1
(43) Pub. Date: Jun. 23, 2022

(54) TRACKING SYSTEM FOR CULTIVATED PRODUCTS AND ASSOCIATED METHODS

(71) Applicant: GLOBAL CANNABIS APPLICATIONS CORP., Vancouver (CA)

(72) Inventor: BRADLEY MOORE, Vancouver (CA)

(21) Appl. No.: 17/456,385

(22) Filed: Nov. 24, 2021

Related U.S. Application Data
(60) Provisional application No. 63/126,555, filed on Dec. 17, 2020.

(51) Int. Cl. A61K 31/352; G16H 70/00; A01G 22/00; G06Q 30/02; H04L 9/32; G06N 20/00

(52) U.S. Cl. CPC A61K 31/352; G16H 70/00; A01G 22/00; H04L 2209/38; H04L 9/3226; G06N 20/00; G06Q 30/0208

(57) ABSTRACT

A tracking system includes a processor, a communications network, a blockchain, and a cultivator device. Growth data on each of the growing states of a cultivated cannabis batch is generated and stored on the blockchain. An intermediary device generates batch segment data having growth data of the cultivated cannabis batch and post-cultivation processing data for that batch segment. Unique identifiers for an end-user product include a link to stored consumer data on the blockchain that includes a) attestation of the authenticity of the end user product, and b) batch segment data for that batch segment to which the end user product belongs. A consumer device scans an end-user unique identifier and retrieves the consumer data from the blockchain and transmits an efficacy value regarding efficacy to the consumer of the end user product.

### PRIORITY APPLICATION(S)

This application is based upon U.S. provisional patent application Ser. No. 63/126,555 filed Dec. 17, 2020, the disclosure which is hereby incorporated by reference in its entirety.

### FIELD OF THE INVENTION

This invention relates to tracking systems, and more particularly, to tracking the growing states of cultivated products and their post processing and related methods.

### BACKGROUND OF THE INVENTION

Tracking systems are used not only for inventory control, but also for many end use applications, including inventory control, minimizing supply chain issues, and tracking commercial, retail, and agricultural products. For example, different growing stages of agricultural products may be tracked to increase growth efficiency. An example includes tracking cannabis products since these products are often illegal or subject to strict regulatory control in many jurisdictions.

The multi-billion dollar global medical cannabis market is growing, but there is a lack of regulatory approved medical cannabis products beyond its basic leaf form. The regulatory process to approve cannabis products is slow and inefficient, and it takes substantial time to generate data for product approvals through clinical studies. Many governments are moving forward with the legalization of medical cannabis, but there is a need to fill the void of medical cannabis data.

Although there is much anecdotal evidence that affirms the efficacy of cannabis in treating conditions from glaucoma to cancer, the federal government has long prevented scientific analysis of these claims, and the strains, delivery, modalities and dosages that best alleviate each ailment are not yet firmly established. Some research has focused on the cannabis plant's positive effects, but it has been limited, and that lack of data leaves cannabis providers, dispensaries, and medical professionals at a loss as to what cannabis products to recommend. Thus, patients often are left to experiment on themselves to discover an effective treatment, which in some cases, may have dangerous consequences.

There is much apprehension from doctors and physicians about which medical cannabis should be recommended and what works best for a specific end-user or patient. Some state laws lay out specific conditions that qualify a patient for medical marijuana access, and the responsibility for actually recommending or prescribing the medical cannabis is left to the physician, who often does not have enough data to recommend medical cannabis or a specific type. This lack of scientific and anecdotal evidence regarding medical cannabis is of great concern to many physicians. Further information to a physician who wants to prescribe a cannabis product is necessary.

For example, different cannabis strains having different concentrations of cannabinoids and terpenes and may produce different end results or medical benefits. The endocannabinoid system plays an important role within the human body, and includes the CB1 and CB2 receptors that are gateways for the cannabinoids to enter the body. These receptors and the effect of cannabis on the body may vary with each individual. Use of cannabis may change the function of these receptors and the chemistry of the human body. The short or long term effects could vary, depending on the individual end-user. Physicians require better data to make better diagnoses and prescriptions, and need the ability to choose cannabis strains and products that would best fit the special needs of their patients. Even retail or dispensary employees with some knowledge of cannabis strains and uses of CBD oil or THC should have access to better data to make better decisions.

### SUMMARY OF THE INVENTION

In general, a tracking system may comprise a processor having an owner host application, a communications network connected to the processor, a blockchain accessible by the owner host application, and a cultivator device connected to the communications network and processor, and configured to generate growth data on each of the growing states of a cultivated cannabis batch grown by a cannabis cultivator and access the blockchain and store the growth data on the blockchain. The cultivator device may be configured to generate a batch unique identifier that links to the growth data stored on the blockchain.

At least one intermediary device may be coupled to the communications network and configured to generate batch segment data and access and store the batch segment data on the blockchain, and generate a plurality of batch segment unique identifiers linking to respective batch segment data on the blockchain for a batch segment that had been split off from the cannabis batch for separate post cultivation processing. The batch segment data may include the growth data of the cultivated cannabis batch to which the batch segment belongs and post cultivation processing data for that batch segment. One of said intermediary devices may be configured to generate end user unique identifiers specific for an end user product that consumers purchase and comprising a link to stored consumer data on the blockchain that includes a) attestation of the authenticity of the end user product, and b) batch segment data for that batch segment to which the end user product belongs. A consumer device may be configured to scan an end user unique identifier and retrieve the consumer data from the blockchain and transmit via the owner host application to the cannabis cultivator of the cannabis batch an efficacy value regarding efficacy to the consumer of the end user product.

A plurality of consumer devices may each be configured to scan an end user unique identifier and transmit an efficacy value. The processor may be configured to determine an average efficacy value per end user product. The consumer data may include data stored on the blockchain of the “best before date” for the end user product. The unique identifiers may comprise QR codes. Upon transmission of the efficacy value, the consumer device may be credited with reward points. In an example, the reward points may comprise a credit of a communications account of the user. The batch segment data may include a THC/CBD concentration of a batch segment.

A method aspect of tracking may comprise loading on a processor an owner host application and connecting a communications network to the processor. The method may include accessing a blockchain by the owner host application, and operating a cultivator device to generate growth data on each of the growing states of a cultivated cannabis batch grown by a cannabis cultivator and accessing the blockchain and storing the growth data on the blockchain. The cultivator device may be configured to generate a batch unique identifier that links to the growth data stored on the blockchain.

The method further includes coupling at least one intermediary device to the communications network and processor, and generating batch segment data and accessing and storing the batch segment data on the blockchain, and generating a plurality of batch segment unique identifiers that link to respective batch segment data on the blockchain for a batch segment that had been split off from the cannabis batch for separate post cultivation processing. The batch segment data may include the growth data of the cultivated cannabis batch to which the batch segment belongs and post cultivation processing data for that batch segment. One of said intermediary devices may be configured to generate end user unique identifiers specific for an end user product that consumers purchase and comprising a link to stored consumer data on the blockchain that includes a) attestation of the authenticity of the end user product, and b) batch segment data for that batch segment to which the end user product belongs.

The method further includes scanning from a consumer device an end user unique identifier and retrieving the consumer data from the blockchain and transmitting via the owner host application to the cannabis cultivator of the cannabis batch an efficacy value regarding efficacy to the consumer of the end user product.

### DESCRIPTION OF THE DRAWINGS

Other objects, features and advantages of the present invention will become apparent from the detailed description of the invention, which follows when considered in light of the accompanying drawings in which:

FIG. 1 is a block diagram of the tracking system in an example embodiment.

FIG. 2 is a block diagram showing an example artificial intelligence model that can be used with the tracking system of FIG. 1.

FIG. 3 is a flowchart showing an example sequence used in assigning a unique identifier to an end user product.

FIG. 4 is a flowchart showing an example sequence of a data flow that may be used with the artificial intelligence model of FIG. 2.

FIG. 5 is a flowchart showing an example sequence for blockchain sorting.

FIG. 6 is schematic diagram of different data blocks that may be used in the blockchain and the tracking system of FIG. 1.

FIG. 7 is another schematic diagram of data blocks for the blockchain similar to that shown in FIG. 6.

FIGS. 8A-8I are a series of interconnected flowcharts showing a high-level sequence of an example for operating the tracking system as described relative to FIG. 10.

FIG. 9 is another schematic diagram of example data blocks such as shown in FIGS. 6 and 7.

FIG. 10 is another block diagram of the tracking system in accordance with a non-limiting example.

### DETAILED DESCRIPTION

The present invention will now be described more fully hereinafter with reference to the accompanying drawings, in which preferred embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art. Like numbers refer to like elements throughout.

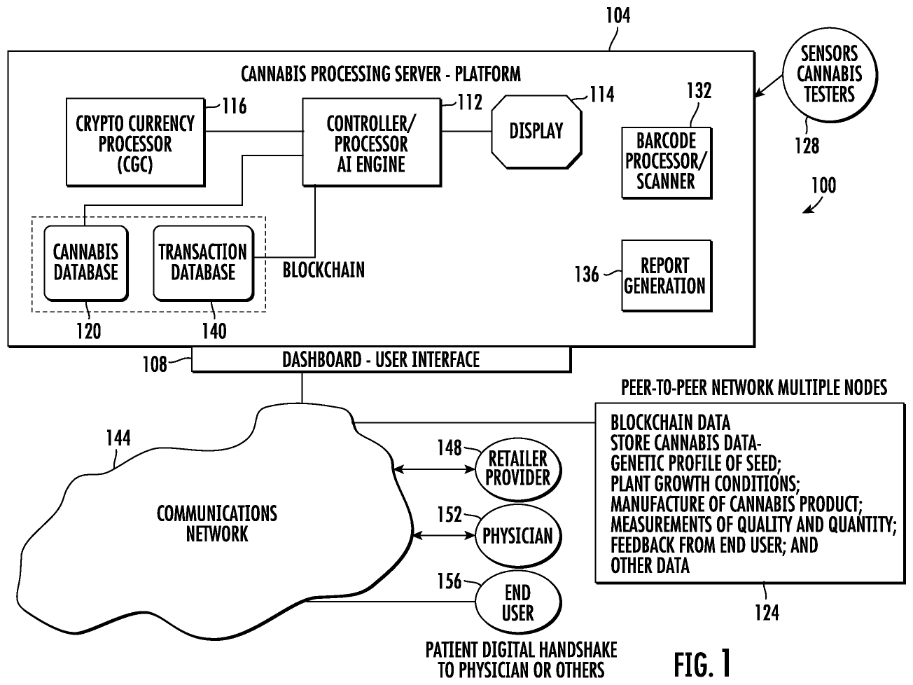

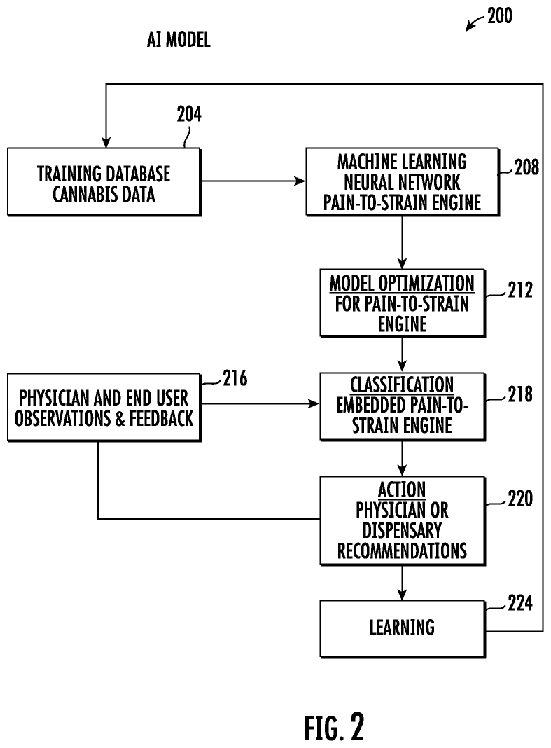

The tracking system as will be described with reference to FIG. 1 is shown generally at 100 and provides a patient care solution that includes a server 104 that operates as a medical cannabis medical platform and covers the full cycle of patient treatment and corresponds it to real-time retail data. The system 100 also offers a complete dashboard as a user interface 108 that displays both greater market data and specific store data leading to better patient outcomes and a healthier business. As part of a user interface and dashboard 108, a product session may display a one-of-a-kind artificial intelligence (AI) based forecast of how certain cannabis products perform in a retail store or dispensary and in the market. The server 104 includes a controller 112 as a processor that also includes artificial intelligence (AI) engine functionality as shown in FIG. 2 and described later.

Product lists from a dispensary or store can be uploaded or available through an API to show what cannabis product is being recommended is in stock, creating a smoother customer experience. The system 100 aggregates patient and customer data about symptoms compared to what other people globally are suffering from, allowing a path for creating effective patient care plans. The API contained on the controller 112 and an associated display 114 connected to the controller as part of a computing device may include promotion effectiveness, where end-users, physicians, dispensaries and providers may find information about how cannabis products are being marketed, e.g., associated promotions, both through business and against other retailers. The API as loaded on the controller 112 may also display on phones or other devices, and the display 114 may include a graphical user interface (GUI) that may include a list of the top symptoms, e.g., six symptoms, that patients globally request treatment. For example, the API may display a left side bar, column, or other GUI listing of the symptoms and other data. Physicians and others will be available to track a patient's progress in treating new symptoms and what others are using as effective answers. A physician may be connected to a patient through a digital handshake, which binds the patient to the physician's or dispensary's store, creating loyalty, better patient care, and real-time communication. A retail location with thousands of handshakes creates a level of insights to (a) provide best possible outcome, and (b) understand the business requirements from stocking to marketing.

A physician or other user may link to a new patient, beginning with an invitation to download the patient application such as using the system 100 shown in FIG. 1. Once the patient has downloaded the application, the patient may be asked to complete a quick and secure sign-up and onboarding process to start tracking symptoms. As part of the onboarding process, the patient may be prompted to provide simple information about symptoms and cannabis use, including how often they use it and their preferred strain, if applicable, including a ratio of THC to CBD, and the type of desired cannabis products if that information is available. This information may be fed anonymously into a “pain to strain” AI engine 112 as part of the controller explained in greater detail relative to FIG. 2, and may be used to build a patient's profile for personalized and accurate recommendations. Once the onboarding is complete, the patient's application is linked to the physician, cannabis dispensary, or other retailer. The patient and others, including a physician and/or retailer or cannabis dispensary, may receive recommended products. For a retailer, the system's recommendations may help better guide and advise patients.

One aspect of the system 100 is the unique cryptocurrency that patients may earn for participating and may use for the cannabis transactions. A patient's account may be managed in a built-in cryptocurrency wallet as a non-limiting example. By tracking sessions in the system 100, patients may follow their treatment and progress.

For example, patients may pick symptoms to track during the onboarding process. Later, this profile may be supplemented with the products purchased at a dispensary. Before a session, the system 100 may ask the patient a few brief questions and when the session begins, the patient records each puff, each vape puff, or each bite for the cannabis product. After the session, the patient is asked to evaluate their post-session feeling: better, the same, or worse. The patient may then view a session summary via the API, which offers insights about their symptoms and treatment progress. Patients may be rewarded with cryptocurrency. In an example, the API may include a promotion tab that offers different promotions from a retailer.

Patients who are linked to a dispensary operated by a retailer or physician may see personalized combination deals for different cannabis products. The controller 112 that includes the AI engine may identify cannabis products based on the store's stock and specific tracked sessions. Likewise, using the controller 112 and its AI engine, the dashboard as part of a user interface 108 may provide product recommendations based on queries about symptoms to help retailer or dispensary workers better serve customers and patients.

As noted before, the tracking system 100 shown in FIG. 1 includes the cannabis processing server 104 as a platform that includes the controller as a processor 112 and operates as an AI engine. The server 104 includes a cryptocurrency processor 116 and may include a cannabis database 120 that may store cannabis data that is also stored in a blockchain 124, such as the genetic profile of the seed, the plant growth conditions, the manufacturing details of the cannabis product, the different measurements of quality and quantity, the feedback from the end-users such as a consumer patient, and other data. Cannabis sensors and cannabis testers 128 may input data to the server 104 regarding the quality of the cannabis. This may include data about desired and undesired components such as CBD, THC, and other additives. The server 104 also may include a bar code and processor/scanner 132 connected thereto that may generate bar codes of the cannabis products. A report generator 136 is operative with the controller 112 as a processor to generate reports that may be printed or displayed to end-users via the dashboard and user interface 108. Different transactions such as between a consumer and retailer or other parties may be stored as records within the transaction database 140.

Because blockchain may be used, the communications network 144 is operative with the different multiple nodes as part of a peer-to-peer network and with the blockchain 124, in an example, which permits a retailer provider 148 or dispensary or other cannabis provider to input and read data. A physician 152 and end-user 156, such as a consumer or patient, may interoperate and a digital handshake occur between the physician and other users.

Referring now to FIG. 2, there is illustrated an artificial intelligence (AI) model generally at 200 that may be used for the controller and AI engine 112 of FIG. 1 and may include a training database 204 that includes stored cannabis growth data and preprocessing data and operative with a machine learning neural network 208, which in this example, could be a pain-to-strain engine as part of the controller 112 in FIG. 1. The data output from the pain-to-strain engine 208 may be used for model optimization for the pain-to-strain engine 212 and include classification 218 of different cannabis data and cannabis products for the pain-to-strain engine 208 with input by physician and end-user observations and feedback 216. An action segment may include the physician or dispensary recommendations 220 with further machine learning 224 and loop back to the training database 204 that includes the cannabis data and other data.

The artificial intelligence model 200 may operate with the training database 204 and blockchain database 124 of FIG. 1 that is a distributed ledger in an example and perform virtual machine logic that may be stored in memory corresponding to the cannabis database 120 within the cannabis processing server 104 of FIG. 1. The blockchain 124 ledger may be a continuously growing ledger adding data that is provided not only by different retailers and providers, physicians and end-users as consumers and patients, but also the quality control of different cannabis products, including genetic profile of seeds, the plant growth conditions, manufacturing requirements, parameters involved with specific cannabis products, and measurements of quality and quantity. Successful therapies may be added to the blockchain 124, as well as unsuccessful therapies and possible therapies. Different contracts, including financial matters between different providers, physicians and retailers, and end-users as patients, may be stored on the blockchain 124. The server 104 and associated processing devices may be formed at field programmable gate arrays, application specific integrated circuits, and include different memory circuits. Other processors may be used. Natural language questions may be used and converted to answers using the machine learning and converted to computer readable data.

The system 100 may include tags, sensors, readers and computer hardware, and associated software that includes tag identification creation, such as bar codes, QR codes, sensor data collectors, data management, and data distribution that are designed to provide assurances to the purchasers of the cannabis products at a wholesale level that the product meets the legally required tracking and quality standards, which are then displayed by retailers at the point of sale.

Cannabis products are legally available for human consumption for several purposes, including but not limited to, medicinal, research and recreational purposes. Undesired and in some cases toxic chemicals, including pesticides and plant growth regulators, are sometimes included in the cannabis products and threaten the health of the consumers. As some cannabis products can be inhaled rather than eaten, any toxins carried by the products have a direct route into the lungs and blood stream of the consumer. Some countries have regulations for controlling the environment where the cannabis plants are grown. Most countries where cannabis products can be legally obtained have no means for ensuring that the plants are grown under controlled environments.

In addition to the natural contamination that may occur during growth of the plants, cannabis and cannabinoid products may be unscrupulously contaminated by using extracts or dried parts of other plants, glass particles, industrial chemicals, sugar or sand, and other micro contaminants. There are no robust integrated systems to ensure that the cannabis products are free of chemical and microbiological contamination, and that the product can be traced as the plant is grown, processed into products, and moved to stores for public consumption. Most consumers do not have access to cannabis or cannabinoid products that have been tested for purity by third party validated labs. The cannabis plant has a complex nature with many unknown variables that are being currently discovered and, in some cases, tested. The ongoing research lacks reliable data on the chemical compound and particular molecules.

Medical cannabinoids and botanical drugs have emerged as a viable means of therapy with growing scientific evidence of therapeutic potential. However, very little work has been accomplished for assuring safe cannabis product development and consumption, and evaluating the direct correlation to the outcomes of diseases and conditions. Various methods of hybridization of the cannabis seed have been used to exploit the variance in cannabinoid profile produced by the plant. However, cannabis product characterization and its connection to disease outcomes have yet to be tracked with precision. Variations in the product due to the nature of it being a plant and growing methodology can lead to inconsistency in cannabinoid products as well as generating other uncertainties in the progeny, such as plant selection, one of the crop improvement selection methods, that cannot be initially predicted. Without a comprehensive verification/validation process, distortion in medical prognosis and diagnosis of providing recommendation or prescribing cannabis for various disease states can arise.

Furthermore, public safety and public trust are the thrusts behind policy and regulation surrounding cannabis. Current platforms lack a comprehensive approach to management of the various aspects of the industry, such as selection of the cannabis seed genetics, managing plant growth conditions, production of products and medicaments with directed safety and therapeutic profile.

Public health and safety risks exist when cannabis or cannabinoid products are not accompanied by adequate information about their sources, manufacturing practices, contents, results of any quality testing and quality assurance procedures. As noted before, toxic chemicals, including pesticides and plant growth regulators, may be part of some cannabis products and threaten the health and safety of the consumers. Additional testing and compliance activities may be needed before public consumption. The cannabis processing server 104 of FIG. 1 may interoperate with cultivators, distributors, retailers and consumers. Cultivators may sample cannabis and cannabinoid products and analyze these products for research to test and set parameters for third parties, such as government agencies to grant awards, and for public safety, quality control and quality assurance purposes. The system 100 may evaluate one or more cannabis products and cannabinoid products for use in a particular industry for research, public use, healthcare, or a combination thereof.

By way of example, the system 100 may include a distributed validated cannabis testing system, including one or more processors or controller 112, an input/output unit such as part of the controller, and configured to communicate with one or more processors, and one or more cannabis databases 120 in communication with the one or more processors to store and associate a plurality of regulatory guidelines with a plurality of measurements of quality and quantity of desired components, such as obtained from a cannabis cultivator or laboratory, and undesired components in a cannabis product. In addition, one or more electronic interfaces, such as the dashboard/user interface 108, may be positioned to display an online user report and define one or more cannabis user interfaces. A non-transitory computer-readable medium as part of the controller 112 may be integral with or positioned in communication with the one or more processors and have one or more computer programs stored thereon.

The programming may include a set of instructions that when executed by one or more processors, perform operations of generating the cannabis user interface 108 to display to a user one or more online cannabis user reports. The cannabis user interface 108 may also allow an input of data associated with the user or with the cannabis product and determine whether the cannabis product meets the regulatory guidelines responsive for receiving the plurality of information associated with the user or with the cannabis product. Information from the one or more cannabis databases 120 may be associated with a plurality of regulatory guidelines and with the plurality of measurements of quality and quantity of desired components and undesired components in a cannabis product, and may output to the one or more user interfaces 108, and may include one or more online cannabis user reports generated by the report generator 136. The cannabis user reports may include one or more of the plurality of information associated with the user or with the cannabis product, and one or more of the plurality of measurements of quality and quantity of desired components and undesired components in a cannabis product for research or for public use.

A distributed validation may integrate profiling and characterizing of the cannabis seed (genetics/multi-omics), the plant/growing process (cultivation), the processing/manufacturing, information technology (IT)/software services, products, and medicaments. The system 100 facilitates safe administration to subjects and provides a means of accessing therapeutic intervention outcomes. The system 100 may track, verify, and validate a cannabis product through a platform with integrated and comprehensive management, i.e., a blockchain system, of that product.

In the current system 100, a data storage instruction may be received as bar code or other identifier symbology via the processor/scanner 132, including data to be stored and an identifier of the data. The blockchain 124 may be identified via the identifier, such as from the bar code scan, and the data on the blockchain may be stored on different blockchain network storage nodes of a data access system, via the a communications network 144, which may include a peer-to-peer network of multiple nodes with different data blocks. The blockchain 124 may be updated by storing the encrypted cannabis data, such as growth data, match segment data, and consumer data in the blockchain 124, and storing the updated data in the particular blockchain network storage node of the system.

The system 100 collects and stores information related to the cannabis product from cultivation, through distribution and manufacturing, retailing and final consumer and provides consumer feedback on the consumption of the end use product across the distributed system that is attested as valid. The system 100 includes the server 104 that associates the plurality of data to a record, which is characterized by a unique identifier, and stores a record into the blockchain 124 for access by one or more authorized users, such as a physician or healthcare provider for an end-user. The cannabis product may be analyzed at different stages such as at cultivation, distribution, manufacturer and retail to determine the quality and quantity of desired components and undesired components in the cannabis product, such as using the sensors and cannabis testers 132.

The determination of the end-user experience may be followed by storage of this data into the blockchain based database 124. The blockchain technology provides cryptographically secure supply chains for uniquely labeled or uniquely identifiable cannabis products such as from a bar code or QR code. Information may be aggregated about cannabis products for quality control and quality assurance purposes. For example, a supplier or consumer of a cannabis product may submit information on the efficacy, potency, quality, and origin of the cannabis product. Samples of the cannabis products may be analyzed for public safety, quality control, and quality assurance purposes. The testing and tracking of cannabis products may be evaluated by health care professionals and patients for quality control, quality assurance, and therapeutic efficacy. The data inputs may be collected and stored on the blockchain 124 using the bar codes or QR codes.

The system 100 as described overcomes the problems associated when legal provided cannabis products take a unique path from their source, through one or more steps of preparation and handling, to wholesale and retail supply chain locations and eventually to the consumer at the final point of sale. A great deal of effort, extensive systems and user convenience may be required. Until implementation of the current system, there were only limited ways to provide customers confidence that the product they are about to purchase is necessarily the product it purports to be.

Correct labeling is a relevant concern for consumers, since consumers want to be confident that the cannabis product they are purchasing is legally complying and fully traceable and verified. The retailers and wholesalers want to know how their product is transported and in what conditions and over what time and the locations and how long the product remained in storage before being made available for sale or used in another product. Consumers likewise require this information and the system 100 provides such remedy.

The blockchain based database 124 with secured collection of product attributes provides advantages for the system 100. Cannabis products may be certified by one or more certifying authorities and subject to additional government requirements. The product tracking involved in the arrangements is not applied to an individual product in all cases. The source of the product via a cannabis cultivator may be known, but some information may not be provided or available to intermediate handlers, such as distributors or manufacturers, since an individual product item may not be labeled. Even if there is a label, the history of the item before and beyond the time of labeling may not be known.

Sensors and cannabis testers 128 are included and with QR codes or other identifiers form a distributed validation system with computer-readable program products, and related devices to track cannabis and cannabinoid products from seed to consumer. The system 100 relies, in one example, upon the blockchain to access and track multiple transactions among various parties involved in manufacture and production of the cannabis products, its subsequent delivery for consumption, and consumption outcomes of individual users. Any trusted individual or company may access the system 100 and blockchain 124 to verify the information associated with any of the transaction records associated with a particular product such as part of the blockchain.

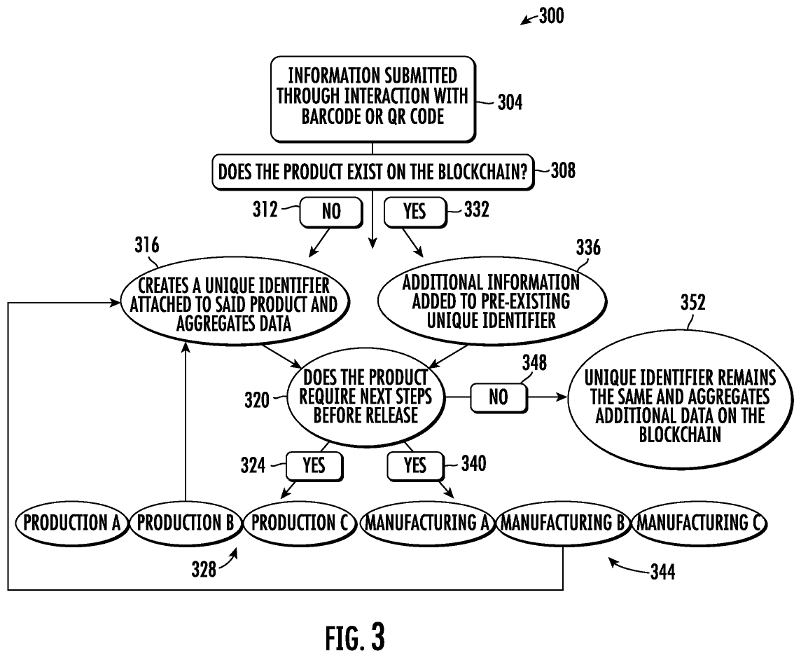

Referring now to the flowchart shown in FIG. 3, there is illustrated generally at 300 a technique for assigning a unique ID such as a bar code or QR code to cannabis products. Information may be submitted via bar code or QR code 304 and the system 100 may determine whether the product exists on the blockchain 124 as shown at 308. If it does not 312, then a unique identifier, such as a bar code or QR code, is created and attached to the product and the data aggregated 316. A determination may be made whether the product requires next steps before release 320, and if yes 324, information such as from different production processes as labeled A, B or C may be input 328. If, on the other hand, the product does exist in the blockchain and indicated at 332, then additional information may be added to the pre-existing unique identifier 336 and a determination made whether the product requires next steps before release 320, and if yes 340, then different manufacturing sequences may be accomplished as shown generally at 344. If not 348, then a unique identifier, such as the QR code, remains the same and aggregates additional data on the blockchain 352.

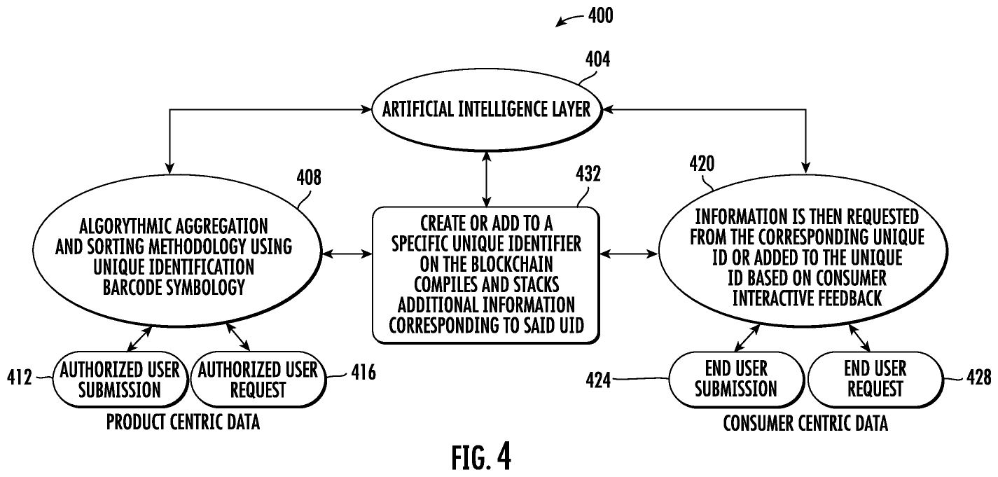

Referring now to FIG. 4, there is illustrated generally at 400 a data flow showing the artificial intelligence (AI) layer 404 as part of the AI engine for the controller 112 that may create or add to a specific unique identifier on the blockchain 124 and compile and stack additional data corresponding to the unique identifier (UID), such as the bar code or QR code. The data and any algorithms may be aggregated and sorted as shown at 408 using the unique identifier, such as the bar code, and include authorized user submissions 412 and authorized user requests 416 for product centric data. Information and other data may be requested from the unique ID such as a QR code and based on consumer interactive feedback 420 for consumer centric data based on data entry, such as an end-user submission 424 and end-user request 428. The authorized user could be a physician, while the end-user could be the patient in this example. Data may create or add to a specific unique identifier, such as the QR code on the blockchain that compiles and stacks additional information corresponding to the unique identifier 432.

Referring again to FIG. 1, the system 100 may manage information related to a cannabis product across a distributed validation system. The system 100 may include enabling a first authorized user to create a first plurality of data containing a genetic profile of a seed used for production of the cannabis product. The system 100 may associate the first plurality of data to a first record, which is identified by a first unique identifier, such as a QR code. The system 100 may include storing the first record into a memory, such as at the server 104 and/or blockchain 124, for access by one or more of a plurality of authorized users using the first unique identifier. The system 100 may include enabling a second authorized user to create a second plurality of data containing plant growth conditions of a crop used for production of the cannabis product. The system 100 may include associating the second plurality of data to a second record which is identified by a second unique identifier. The system 100 may include storing the second record into the memory, such as block chain 124, for access by the one or more of the plurality of authorized users using the second unique identifier. The system 100 may further include enabling a third authorized user to create a third plurality of data containing manufacturing information for production of the cannabis product. The system 100 may include associating the third plurality of data to a third record which is identified by a third unique identifier. The system 100 may include storing the third record into the memory, such as the blockchain 124, for access by the one or more of the plurality of authorized users using the third unique identifier.

The system 100 may include analyzing the cannabis product to determine quality and quantity of desired components and undesired components in the cannabis product using one or more of: cannabinoid profiling, microbiological testing, analytical testing, pathogen testing, quality control testing, and quality assurance testing. The system 100 may include determining concentration of one or more cannabinoids in the cannabis product.

The system 100 may include enabling an authorized user to create a plurality of data containing genetic profile of a seed, plant growth conditions of a crop, and manufacturing information used for production of the cannabis product, and measurements of quality and quantity of desired components and undesired components in the cannabis product. This may also include the feedback from an end-user creating a respective dataset.

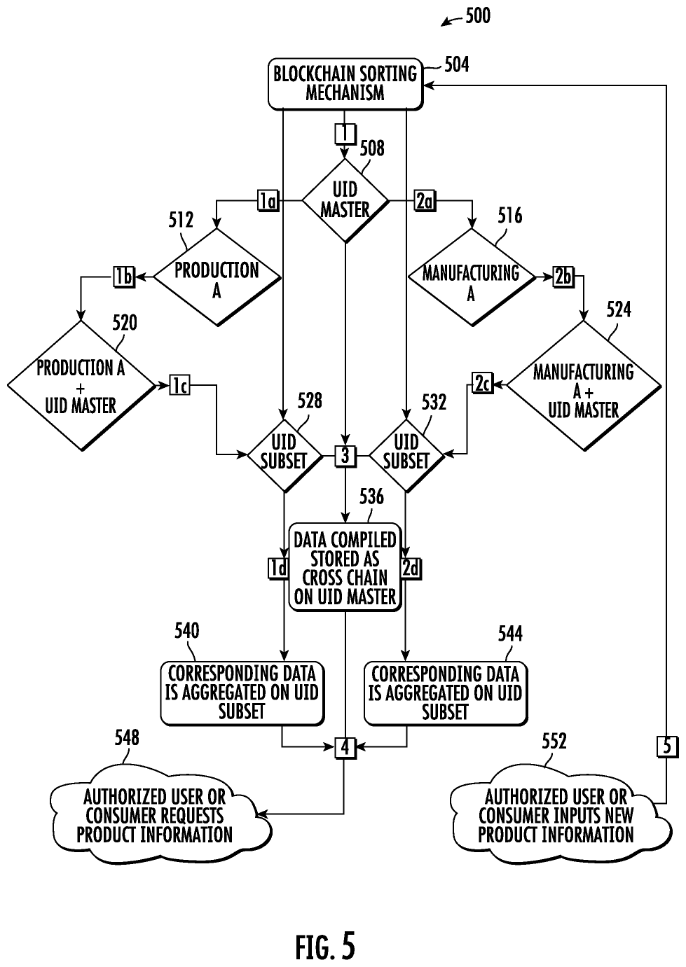

Referring now to FIG. 5, there is illustrated generally at 500 a block diagram of a blockchain sorting mechanism 504 that includes first the UID master 508, such as generated at the server 104, with production 512 and manufacturing 516 sequences illustrated and the production with UID master 520 and manufacturing with UID master 524 and their subsets 528, 532, including data as compiled and stored as a cross-chain on a UID master 536 with corresponding data aggregated at different subsets 540, 544. For example, an authorized user, such as a physician or consumer, may request product information 548 or input new product information 552, which is then compiled via the blockchain sorting mechanism.

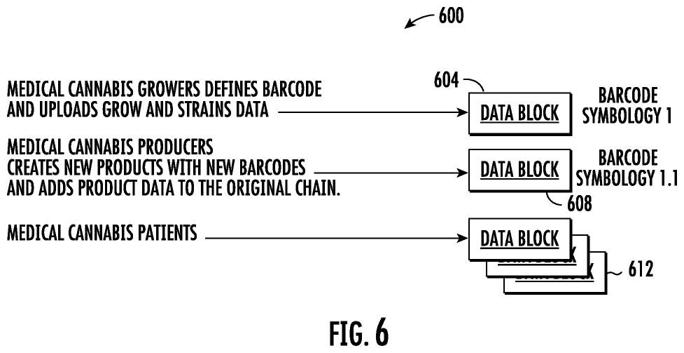

Referring now to FIG. 6, there is illustrated data aggregation generally at 600 where medical cannabis growers may define a bar code and upload growth and strain data as part of a data block 604 for the blockchain with a unique identifier, such as a bar code or QR code. Medical cannabis producers may also create new products having new bar codes and add product data to the original blockchain as a second data block 608. Medical cannabis patients and providers may also add to the blockchain with their various inputs as third data blocks 612.

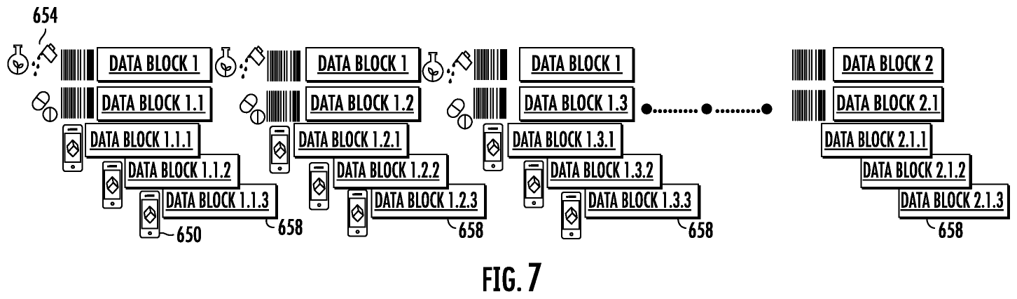

As shown in the block diagram of FIG. 7, data may be input into the blockchain 124 via portable communication devices, such as a portable phone or other devices 650, and via testing and sensors 654 with bar codes or QR codes as pointers to the data. A cannabis product may be tested and that information entered to the blockchain and a cannabis product scanned with a bar code and later users input data and the data maintained in the blockchain via the data blocks 658.

The system 100 as described may improve patient outcomes and grow a retailer's or cannabis provider's business using data insights created through smart technologies. The system 100 may include an integrated mobile and desktop solution provided by the server 104. A rewards program may use the utility cryptocurrency as rewards. It may provide a marketplace for trading data and having insights between medical cannabis retailers using the server 104. The system 100 is relevant for medical cannabis retail outlets that operate in both physical locations and on-line systems. The system 100 may be integrated into an online store and different membership levels may be chosen. Raw data may be collected from patients and incorporated into the cannabis database 120 as part of the server 104 and within the blockchain 124 network. The system may use machine learning analysis by incorporating the AI engine as part of the controller 112. Personal data from patients may be stripped away and only anonymous health data and results captured, while findings from different patient interactions on an anonymous level may be available to community members, depending on different membership levels.

The pain-to-strain machine learning engine as part of the AI model may drive better health outcomes and allow retail dispensaries to focus on the success of their business. The dashboard and user interface 108 may be applicable for retailers and provides a product recommendation portal, which via the server 104 may analyze the patient profile and medical information to make a treatment recommendation. An in-store dashboard may allow patients to download the application on a mobile phone, for example, or other device. Patients may confirm which of the retailer's products they purchased and begin tracking treatment journeys. Some membership levels may be accomplished, such as a “freemium” option with limited reports or a premium data membership level that allows patient care on a one-to-one basis and allows recommendations per ailment and product insights of multiple reports based on numbers of different patients. A stand-alone system may be accessed through an on-line dashboard using the computer or tablet or the internet and a browser.

Different rewards can be provided such as the cryptocurrency as described above, as an incentive tool to build loyalty, acquire new patients, and retain existing patients. Consumers may be rewarded with a credit to a phone account. Retailers may trade data insight reports in exchange for cryptocurrency and by buying and selling reports and data insights. Retailers may earn the cryptocurrency, which can be used to drive marketing and promotion programs to increase sales or buy reports and data insights.

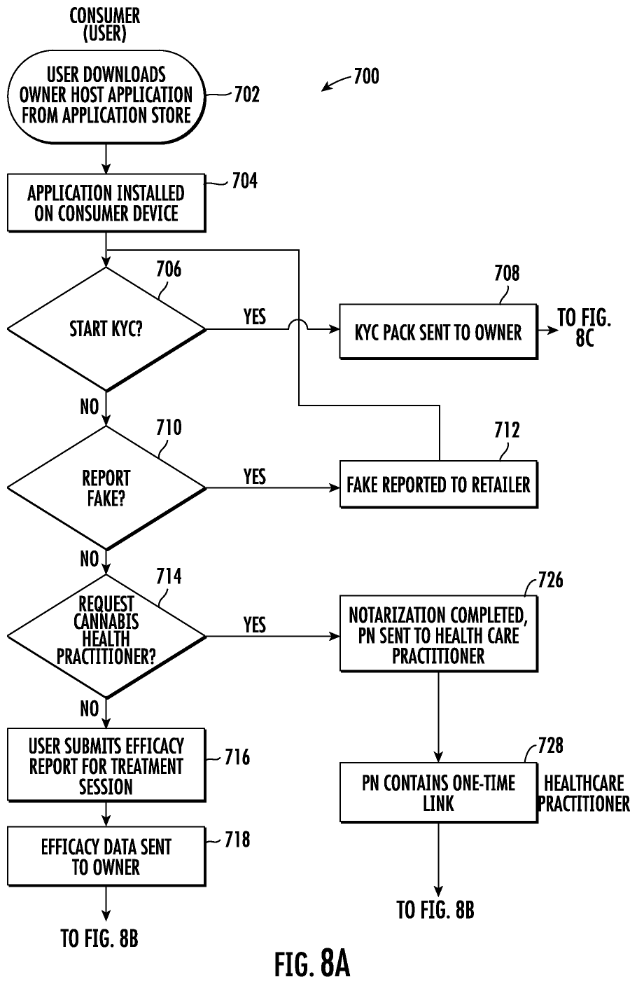

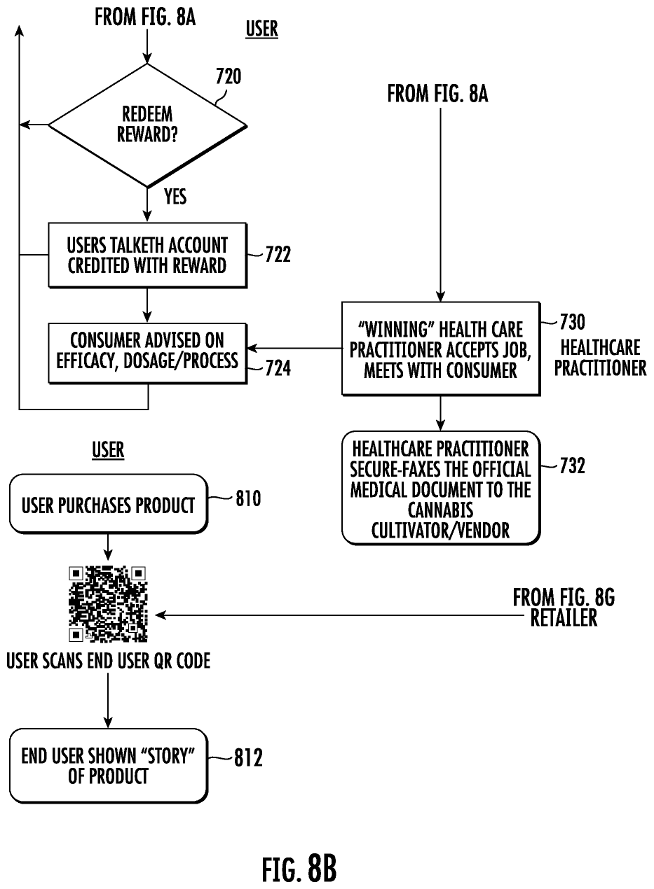

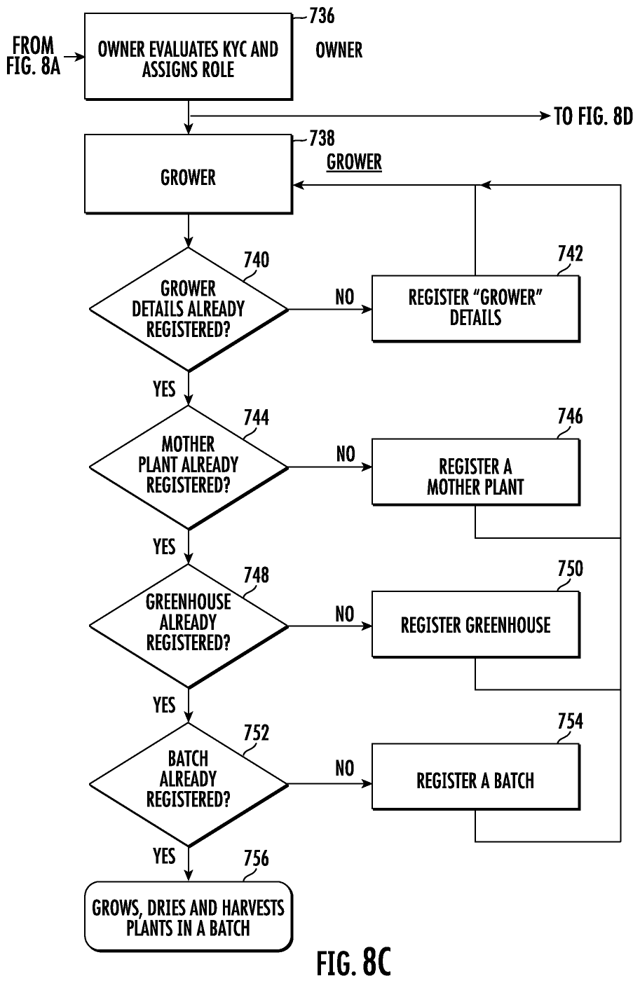

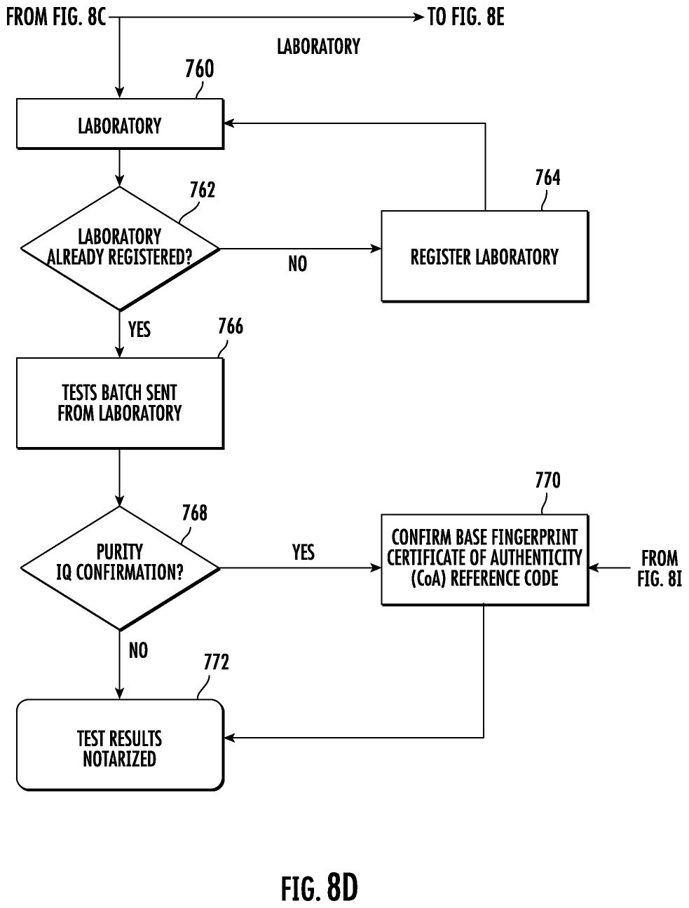

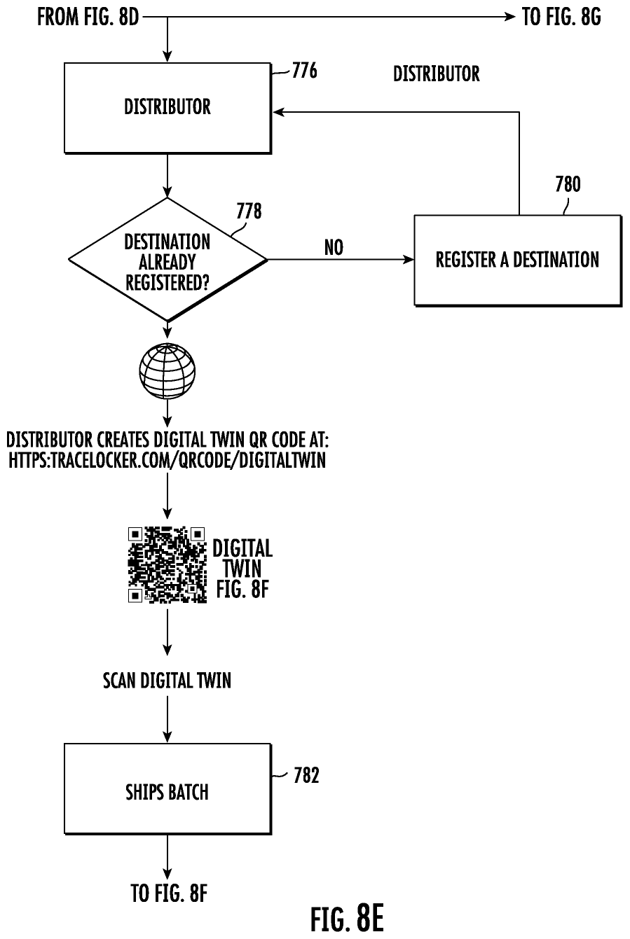

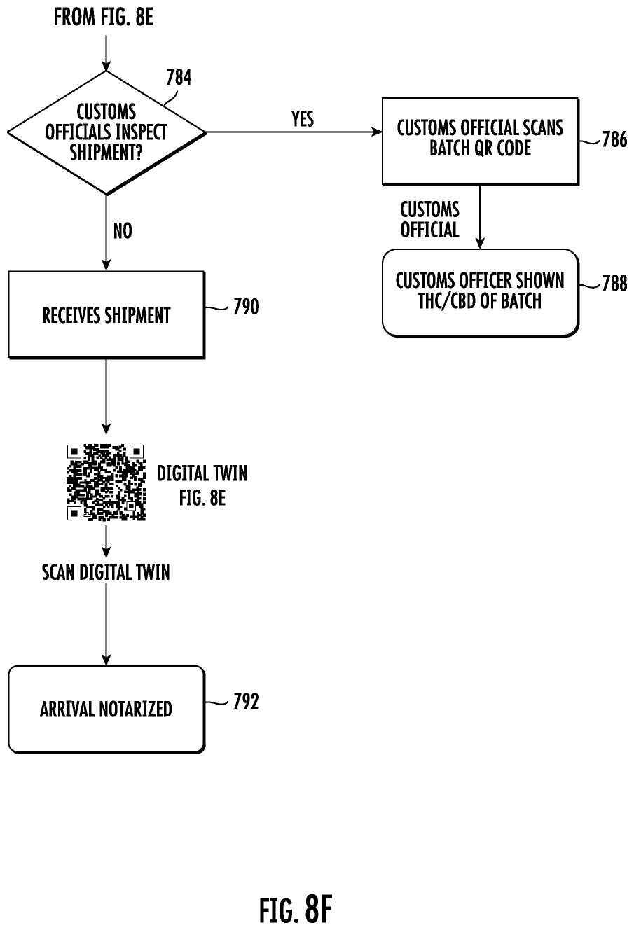

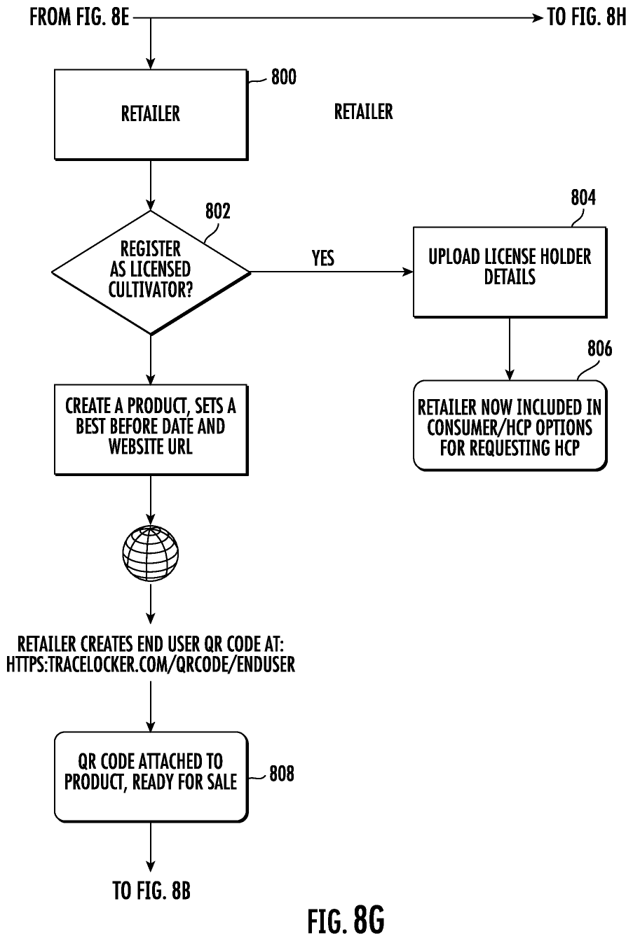

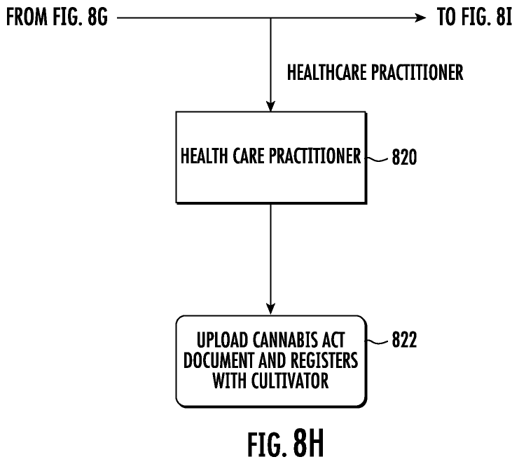

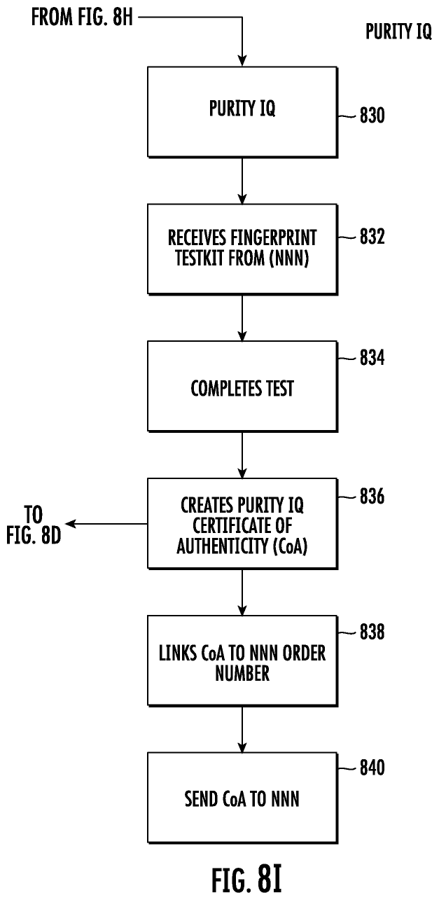

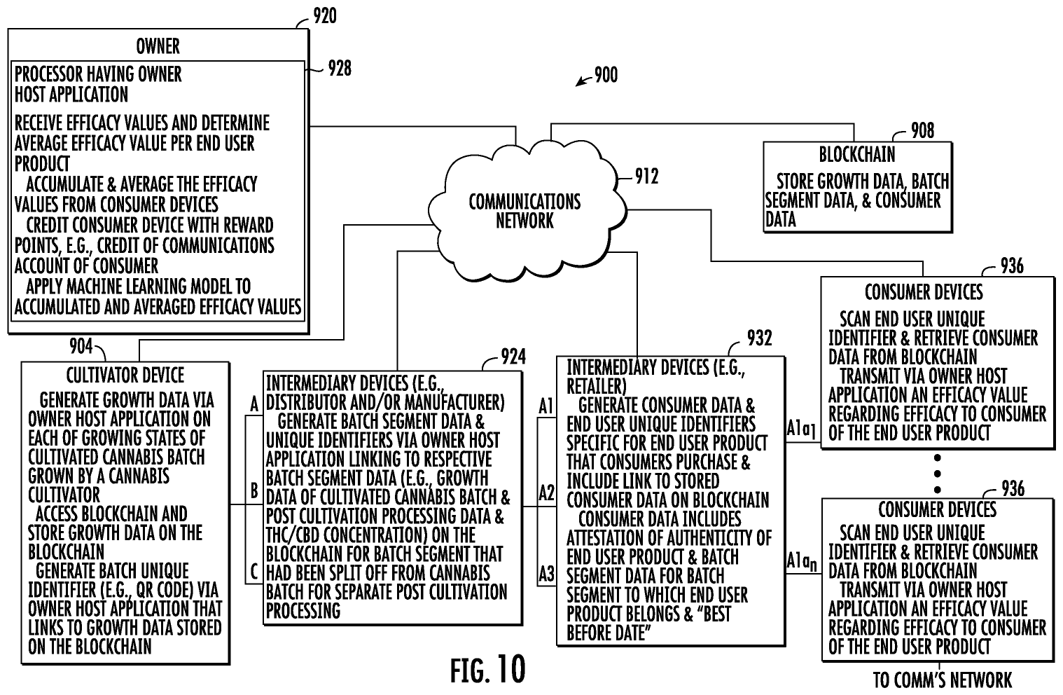

Referring now to FIGS. 8A through 8I, there are illustrated a series of interconnected flowcharts showing a high-level sequence starting at 700 for operating the tracking system as described relative to FIG. 10. For a consumer or user, the user may download the owner host application from an application store 702 and the application is installed on the consumer device 704 as shown in FIG. 8A. A know your client (KYC) 706 may be started and if it is, a KYC pack is sent to the owner 708 with a process described relative to FIG. 8C. A determination is made whether a fake should be reported 710, and if yes, the fake is reported to the retailer 712 and the process loops back. For example, a consumer may scan a QR code and that scanning may bring up an image of a correct cannabis container bottle or other end-user product and a determination can be made from the pictures of the container or serial numbers whether the product on the shelf of a retailer is real or fake. The user as a consumer may request a cannabis health practitioner 714, and if not, the user may submit efficacy report for a treatment session 716 and the efficacy data is sent to the owner 718. Once the efficacy data is sent to the owner, the user may redeem a reward 720 such as a talketh account of the user in Canada, for example, that is credited with the reward 722 and the consumer is advised on the efficacy, dosage/process 724. The user submits the efficacy report after using the product and determining if it had efficacy for the use by the consumer who used the cannabis end use product.

If the user requested the cannabis health practitioner at 714, a notarization may be completed and a PN sent to the healthcare practitioner 726 where the PN contains a one-time link 728. The “winning” healthcare practitioner may accept the job with the consumer and meets with the consumer 730 and the healthcare practitioner may fax a secure document or other official medical document to the cannabis cultivator/vendor 732 so that feedback may help the cannabis cultivator determine the efficacy of the product and type of growing stages that may be used with the specific end-use cannabis product.

As shown in FIG. 8C, the owner may evaluate the KYC and assigns a role 736. The grower 738 has specific requirements and the grower details should be registered 740, and if not, then the grower details are registered 742. The grower details may include the different stages of the growth process. For example, the mother plant should already be registered 744, and if not, then the mother plant is registered 746. The greenhouse should be registered 748, and if not, the greenhouse is registered 750. Batches are registered 752, and if not, then the batches are registered 754, such as an a batch corresponding to a first batch. The registration of the batch may include data regarding the growth, drying, and harvesting of plants in the batch 756.

The laboratory 760 also has specifications and the laboratory should be registered 762, and if not, then the laboratory is registered 764. Test batches may be sent from the laboratory 766 and the purity IQ of a batch may be confirmed 768, and if yes, the base fingerprint is confirmed with the certificate of authenticity (CoA) reference code and test results are notarized 772.

As shown in FIG. 8E, the distributor 776 process is illustrated and a determination is made whether the destination is already registered 778, and if not, then the destination is registered 780. If the destination is registered, the distributor may create a digital twin QR code at a website that may be accessed from their distributor device such as a personal phone, laptop, computer, or other device. The digital twin is scanned and the batch is shipped 782. Customs officials (FIG. 8F) may be involved and a determination is made whether they inspect the shipment 784, and if yes, then the customs official scans the batch QR code 786 and the particular information regarding the batch is brought up such as may be stored on the blockchain and some history of the growth stages may be brought forth as well as the concentration of the THC and CBD of the batch 788. The shipment may be received 790 at a manufacturer or retailer and the digital twin is scanned to attest to the authenticity, such as via a certificate of authenticity and the arrival notarized 792 and may also be entered into the blockchain.

As shown in FIG. 8G, the retailer 800 process is shown and a determination is made whether the retailer has been registered as a licensed cultivator 802, and if yes, the license holder details are uploaded 804 and the retailer is now included in the consumer and the healthcare practitioner options for requesting a healthcare practitioner 806. There may be some cultivators that also operate as retailers themselves without going through a distribution process with the distributor and a retailer such as a registered pharmacy. The retailer may create a product and sets a best before date and a website URL so that the end product may be scanned at the retailer and a link to a website made so information may be brought up and any relative information from the blockchain obtained, if necessary. The retailer may create an end-user QR code 808 and the QR code is attached to the product ready for sale.

As shown in FIG. 8B, a user or consumer purchases the product 810 and scans the end-user QR code and the end-user is shown the “story” of the product 812 that may include some limited information about the growth stages and the manufacturing process steps, as well as a picture and other information to attest to the validity of the product sold at the retailer.

Referring now to FIG. 8H, the healthcare practitioner 820 is illustrated and the cannabis act is uploaded with the document and registers with the cultivator 822.

As shown in FIG. 8I, a purity IQ process 830 is illustrated where the fingerprint test kit may be received from a source labeled as in NNN 832 and the test completed 834 and the purity IQ certificate of authenticity (CoA) is created 836 and the certificate of authenticity is linked to an NNN order number 838 and the certificate of authenticity is sent to NNN corresponding to the test kit originator 840. Once the purity IQ certificate of authenticity is created, the base fingerprint certificate of authenticity reference code is confirmed as shown in FIG. 8E as part of the laboratory process.

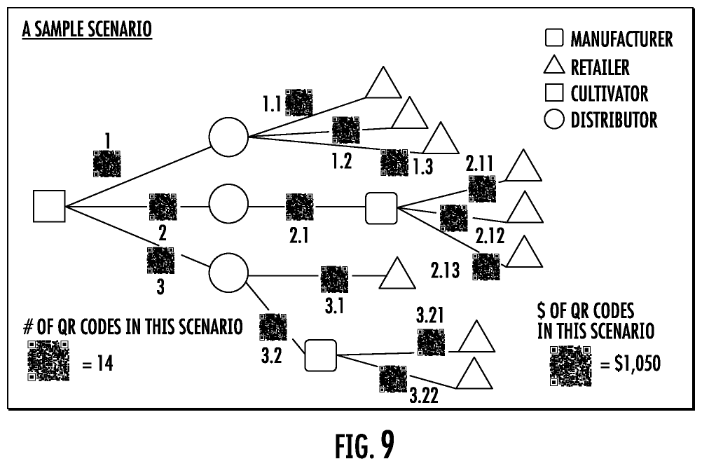

FIG. 9 illustrates a sample scenario with different QR codes and illustrates how the cultivator may start and have different QR codes for different batches of cannabis product in this example, followed by a distributor that then does further QR codes such as 1.1, 1.2, and 1.3 for the retailer. The center path shown 2.1 shows the manufacturer that then issues the different QR codes 2.11, 2.12, 2.13 for the retailer. Similar instances are shown with the third distributor and the different retailers and manufacturers.

The blockchain allows a consumer to complete a relationship between themselves and a cultivator because trust is earned by the cultivator through the transparency of the growing data that is attested. The consumer experience attestation drives all understanding and improvements for all stakeholders. A more normal database may not work as efficiently as the blockchain because what is on it is not inherently visible nor secure.

The blockchain creates a natural feedback loop as a byproduct of its design. When dealing with a plant unique organism, the feedback loop per experience helps achieve enough points to establish an average efficacy, similar to the goal of clinical studies. A key relationship may exist between the cultivator and a consumer/patient because fundamentally without them, there is no requirement for anyone else, i.e., a manufacturer, distributor, retailer, medical practitioner, or similar individual to be involved in the nature of the plant as a unique and individual batch may be a fundamental driver behind the logic.

As shown in FIG. 9, a batch QR code may be a first code generated and designed to represent each of the growing steps attested on the blockchain up to, but not including shipment. Upon receipt of a product, the different distributors and/or manufacturers may then create another QR code or series or codes depending on what or how they treat the product up to, but not including shipment to different retailers. From there, depending on the treatment by a specific retailer X, a number of QR codes may be generated. There may always be different variations, but the cultivation practice per batch is represented by only one QR code that is now diverged into many unique paths represented by new QR codes for the grams of cannabis as they moved towards the consumer. This data is going in one blockchain.

When a consumer uses one of those grams, the consumer may attest their experience onto the blockchain. This means, for example, that there may be 500 bottles in a retail location with all the same QR code, but once a consumer chooses to download the owner application and attest their experience with that 1 gram on the blockchain, there is now a direct connection via this data between the cultivator and the consumer. The benefits in power of these individual multiple data streams are substantial in terms of what can be interpreted and used. For cultivators, this builds a unique relationship on a per consumer basis, creating a feedback loop that is 100% secure in which better product decisions including cultivation and marketing may be made. This may generate more sales without including customer acquisition costs. Consumers may confidentially use a product because the immutable data of both cultivation and other patient experiences is easily accessible through the scan of a QR code. When a consumer chooses to input their own experience, they are participating at a social level, which is an important part of today's consumer mindset. A key to achieving these benefits is full attestation from cultivation to consumption. Ease of use through simple mobile phone technology may be used for all stakeholders, including the cultivator, manufacturer/distributor, retailer, regulator, and consumer that creates adoption, but also incorporates the sophisticated nature of the blockchain functionality.

Referring now to FIG. 10, there is illustrated generally at 900 a block diagram of the tracking system in accordance with a non-limiting example. Different devices are illustrated, including a cultivator device 904 that generates growth data via the owner host application on each of the growing states of the cultivated cannabis batch grown by a cannabis cultivator. The cultivator device 904 may access the blockchain 908 via a communications network 912 and store growth data on the blockchain. The cultivator device 904 may also generate batch unique identifiers such as QR codes via the owner host application that links the growth data stored in the blockchain. The cultivator device 904 may be a small portable communications device, such as an iPhone or other device in which the cannabis cultivator may take snapshots and photographs of cannabis plants and generate data regarding the growth of a particular batch and enter that data onto predetermined forms such as prepared by the owner 920 or make notes.

As illustrated, the batch may be split into batch segments A, B, and C and one batch sent to an intermediary device 924 such as a distributor and/or manufacturer. Batch segment data and unique identifiers may be generated via the owner host application that is loaded on a processor or controller 928 at the owner 920 and links to respective batch segment data, such as the growth data of a cultivated cannabis batch and any post-cultivation processing data that may include the THC or CBD concentration that may be added or modified at the distributor and/or manufacturer. This data may be stored on the blockchain 908 for the batch segment that is has been split off from the cannabis batch for separate post-cultivation processing. For example, batch A may be formulated into a skin cream while batch B may be formulated into a solution that is bottled for consumption. Batch A received at an intermediary device 924 and formulated into different end-user products as A1, A2, and A3 products. Even at the retailer 932, the device may generate consumer data and end-user unique identifiers as a QR code specific for the end-user product that consumers purchase and include a link to stored consumer data on the blockchain 908. The consumer data may include attestation of authenticity of the end-user product such as pictures of what might appear on the retailer's shelf and batch segment data for the batch segment to which the end-user product belongs and a “best before date.”

The end product may be purchased by consumers that each have a consumer device 936 and may scan the end-user unique identifier and retrieve consumer data from the blockchain 908. The consumer 936 may transmit via the owner host application and efficacy value regarding efficacy to the consumer of the end-user product. The owner 920 includes the processor 928 that incorporates the owner host application and receives the efficacy values and determines average efficacy value per end-user product. The efficacy values from consumer devices 936 may be accumulated and averaged and a machine learning model as described above may be applied to determine what cultivation practices may be changed to better adapt certain cultivation practices and growth stages to a particular type of batch.

A consumer device 936 can also be credited with reward points such as credit of the communications account of the consumer. The processor 928 may access the blockchain 908 to store growth data, batch segment data, and consumer data. The owner 920 may access the blockchain 908 via the communications network 912. The cultivator device 904, intermediary devices 924, 932 and consumer devices 936 may all be different types of portable or stationary devices, with most use intended as portable phone devices.

The machine learning model may incorporate artificial intelligence algorithms and may include a prediction model that includes a regression model having a moving window that takes into account mean, standard deviation, skewness, kurtosis, and median. There may be values between 0 and 1 as an output relating to how growth standards can vary, with 0 indicating that no changes are necessary for a cultivator, and a 1 indicating that major changes should be made, e.g., the different growth cycles and parameters of cultivation. These could include changes in temperature, moisture, irrigation, greenhouse environment, and if a greenhouse is used, and other factors. Even position of certain cannabis plants in a greenhouse or field may be determined and processed as an output value.

Many modifications and other embodiments of the invention will come to the mind of one skilled in the art having the benefit of the teachings presented in the foregoing descriptions and the associated drawings. Therefore, it is to be understood that the invention is not to be limited to the specific embodiments disclosed, and that the modifications and embodiments are intended to be included within the scope of the dependent claims.

1. A tracking system, comprising:
a processor having an owner host application;
a communications network connected to the processor;
a blockchain accessible by the owner host application;
a cultivator device connected to the communications network and processor, and configured to generate growth data on each of the growing states of a cultivated cannabis batch grown by a cannabis cultivator and access the blockchain and store the growth data on the blockchain, wherein the cultivator device is configured to generate a batch unique identifier that links to the growth data stored on the blockchain;
at least one intermediary device coupled to the communications network and configured to generate batch segment data and access and store the batch segment data on the blockchain and generate a plurality of batch segment unique identifiers linking to respective batch segment data on the blockchain for a batch segment that had been split off from the cannabis batch for separate post cultivation processing, and wherein the batch segment data includes the growth data of the cultivated cannabis batch to which the batch segment belongs and post cultivation processing data for that batch segment, wherein one of said intermediary devices is configured to generate end user unique identifiers specific for an end user product that consumers purchase and comprising a link to stored consumer data on the blockchain that includes a) attestation of the authenticity of the end user product, and b) batch segment data for that batch segment to which the end user product belongs; and
a consumer device configured to scan an end user unique identifier and retrieve the consumer data from the blockchain and transmit via the owner host application to the cannabis cultivator of the cannabis batch an efficacy value regarding efficacy to the consumer of the end user product.

2. The tracking system of claim 1, comprising a plurality of consumer devices, each configured to scan an end user unique identifier and transmit an efficacy value.

3. The tracking system of claim 1, wherein said processor is configured to determine an average efficacy value per end user product.

4. The tracking system of claim 1, wherein said consumer data includes data stored on the blockchain of the “best before date” for the end user product.

5. The tracking system of claim 1, wherein the unique identifiers comprise QR codes.

6. The tracking system of claim 1, wherein upon transmission of the efficacy value, the consumer device is credited with reward points.

7. The tracking system of claim 6, wherein the reward points comprise a credit of a communications account of the user.

8. The tracking system of claim 1, wherein the batch segment data includes a THC/CBD concentration of a batch segment.

9. A tracking system, comprising:
a processor having an owner host application and machine learning module;
a communications network connected to the processor;
a blockchain accessible by the owner host application;
a cultivator device connected to the communications network and processor and configured to generate growth data on each of the growing states of a cultivated cannabis batch grown by a cannabis cultivator and access the blockchain and store the growth data on the blockchain, wherein the cultivator device is configured to generate a batch unique identifier that links to the growth data stored on the blockchain;
at least one intermediary device coupled to the communications network and configured to generate batch segment data and access and store the batch segment data on the blockchain and generate a plurality of batch segment unique identifiers linking to respective batch segment data on the blockchain for a batch segment that had been split off from the cannabis batch for separate post cultivation processing, and wherein the batch segment data includes the growth data of the cultivated cannabis batch to which the batch segment belongs and post cultivation processing data for that batch segment, wherein one of said intermediary devices is configured to generate end user unique identifiers specific for an end user product that consumers purchase and comprising a link to stored consumer data on the blockchain that includes a) attestation of the authenticity of the end user product, and b) batch segment data for that batch segment to which the end user product belongs; and
a plurality of consumer devices configured to scan an end user unique identifier and retrieve the consumer data from the blockchain and transmit via the owner host application to the cannabis cultivator of the cannabis batch an efficacy value regarding efficacy to the consumer of the end user product, wherein said processor is configured to accumulate and average the efficacy values received from said plurality of consumer devices and apply a machine learning model to the accumulated and averaged efficacy values.

10. The tracking system of claim 9, wherein said processor is configured to determine an average efficacy value per end user product.

11. The tracking system of claim 9, wherein said consumer data includes data stored on the blockchain of the “best before date” for the end user product.

12. The tracking system of claim 9, wherein the unique identifiers comprise QR codes.

13. The tracking system of claim 9, wherein upon transmission of the efficacy value, the consumer device is credited with reward points.

14. The tracking system of claim 13, wherein the reward points comprise a credit of a communications account of the user.

15. The tracking system of claim 9, wherein the batch segment data includes a THC/CBD concentration of a batch segment.

16. A method of tracking, comprising:
loading on a processor an owner host application;
connecting a communications network to the processor;
accessing a blockchain by the owner host application;
operating a cultivator device to generate growth data on each of the growing states of a cultivated cannabis batch grown by a cannabis cultivator and accessing the blockchain and storing the growth data on the blockchain, wherein the cultivator device is configured to generate a batch unique identifier that links to the growth data stored on the blockchain;
coupling at least one intermediary device to the communications network and processor and generating batch segment data and accessing and storing the batch segment data on the blockchain and generating a plurality of batch segment unique identifiers that link to respective batch segment data on the blockchain for a batch segment that had been split off from the cannabis batch for separate post cultivation processing, and wherein the batch segment data includes the growth data of the cultivated cannabis batch to which the batch segment belongs and post cultivation processing data for that batch segment, wherein one of said intermediary devices is configured to generate end user unique identifiers specific for an end user product that consumers purchase and comprising a link to stored consumer data on the blockchain that includes a) attestation of the authenticity of the end user product, and b) batch segment data for that batch segment to which the end user product belongs; and
scanning from a consumer device an end user unique identifier and retrieving the consumer data from the blockchain and transmitting via the owner host application to the cannabis cultivator of the cannabis batch an efficacy value regarding efficacy to the consumer of the end user product.

17. The method of claim 16, comprising a plurality of consumer devices, each configured to scan an end user unique identifier and transmit an efficacy value, and accumulating and averaging the efficacy values received from said plurality of consumer devices and applying a machine learning model to the accumulated and averaged efficacy values.

18. The method of claim 16, wherein said processor is configured to determine an average efficacy value per end user product.

19. The method of claim 16, wherein upon transmission of the efficacy value, the consumer device is credited with reward points.

20. The method of claim 19, wherein the reward points comprise a credit of a communications account of the user.
